(19) United States
(12) Patent Application Publication
(10) Pub. No.: US 2012/0271409 A1
(43) Pub. Date: Oct. 25, 2012

(54) Helical Radiopaque Marker

(75) Inventor: Walter Bruszewski, Windsor, CA (US)

(73) Assignee: Medtronic Vascular, Inc., Santa Rosa, CA (US)

(21) Appl. No.: 13/093,165

(22) Filed: Apr. 25, 2011

(51) Int. Cl. A61F 2/82

(52) U.S. Cl. 623/1.34

(57) ABSTRACT

A radiopaque marker includes a core wire having a proximal portion and a distal portion, and a coil wrapped around the distal portion of the core wire. The core wire is formed from a shape memory material and the coil is formed from a radiopaque material. The radiopaque marker includes a delivery configuration wherein the radiopaque marker is substantially elongated and a deployed configuration wherein the distal portion of the raadiopaque marker forms a substantially helical tube.

### TECHNICAL FIELD

This invention relates generally to a helical radiopaque marker and method of delivering and using such a helical radiopaque marker.

### BACKGROUND

Endovascular aneurysmal exclusion is an evolving method for treating arterial aneurysmal disease. Aneurysmal disease causes the weakening and radial distention of a segment of a vessel, in particular, an artery. This arterial distention results in the development of an aneurysm, i.e., a bulging at the affected arterial segment.

An aneurysm is at risk of rupture resulting in extravasation of blood into, for example, the peritoneal cavity or into tissue surrounding the diseased artery. The goal of endovascular aneurysmal exclusion is to exclude from the interior of the aneurysm, i.e. aneurysmal sac, all blood flow, thereby reducing the risk of aneurysm rupture requiring invasive surgical intervention.

One procedure developed to accomplish this goal entails providing an alternate conduit effectively internally lining the affected artery with a biocompatible graft material. The graft material is configured in a generally tubular shape spanning the aneurysm (intra-aneurysmal). Stents are generally attached to the graft material to couple the graft material to the artery, establishing a substantially fluid-tight seal above and below the distended aneurysmal segment at graft/artery interfaces.

Endoluminal stent grafts are positioned and deployed within the affected artery through insertion catheters by percutaneous procedures well known to those of skill in the art. Once deployed, an endoluminal stent graft provides an alternate conduit for blood flow and, at the same time, prevents the flow of blood into the aneurysmal sac. Endoluminal stent grafts provide a generally effective means to exclude blood flow from aneurysms.

One problem in present stent graft designs is the need to fix the proximal spring stent superior to the renal arteries and superior mesenteric artery when the only region suitable for sealing is superior to these visceral arteries. An estimated ten percent of abdominal aortic aneurysm cases amenable to endovascular repair require suprarenal fixation, cutting off blood to the kidneys and intestine. One proposed solution to this problem has been to provide branched conduits from the stent graft in the aorta to perfuse the renal arteries and superior mesenteric artery.

Unfortunately, the anatomy of the branching of the renal arteries and superior mesenteric artery varies from patient to patient. The axial location, axial angle, and radial angle of the branch vessels all can vary. One approach to this problem has been to provide pre-fenestrated primary stent graft. However, properly aligning the fenestrations with the branch vessels can be difficult.

Another approach to the problem of variable anatomy has been to fenestrate the graft material in situ after the primary stent graft has been deployed, forming a fenestration to provide a passage between the primary stent graft lumen and the branch vessels. The general approach has been to pierce the graft material at the location of the branch vessel to be perfused and to work the hole until it is the size desired. In one case, a needle is used to pierce the graft material and a larger needle used to dilate the needle hole. A balloon is then used to enlarge the dilated hole to a final diameter. A covered stent can be deployed in the hole to provide a flow path between the stent graft lumen and the visceral artery, and to maintain patency of the branch vessel.

One difficulty with in situ fenestration is the amount of force required to dilate the needle hole. The graft material is tough so that excessive axial force is required to dilate the needle hole. This reduces the control of the attending physician and can even result in inadvertent puncture of the vessel wall with the dilator if a slip should occur. Further, precision alignment of the puncture device with the axial location, axial angle, and radial angle of the branch vessel is required to prevent inadvertent puncture of the vessel wall.

Thus, it is important to visualize the position, orientation, and overall geometry of the target branch vessels relative to the main vessel in order to properly align fenestrations through the primary graft with the branch vessels, or to safely create an in situ fenestration in the primary stent graft that is aligned with the branch vessel. However, typical visualization techniques during an endoluminal stent graft procedure are limited. In particular, in an endoluminal stent graft procedure, an angiogram (fluoroscopy with contrast media) is taken prior to delivery and deployment of the stent graft. An angiogram enables a detailed image of the vessels, such as the abdominal aorta and the renal arteries. However, once the procedure for delivery and deployment begins, further images are normally only taken without contrast media, thereby reducing the quality of the image. Further, complications due to contrast media nephrotoxicity may contraindicate the use of contrast media. Further, with conventional angiogram/fluoroscopy techniques, three-dimensional visualization in real on near real is not possible.

Accordingly, a device and method that permits improved visualization of the position, orientation, and overall geometry of a vessel during an endoluminal stent graft delivery and deployment procedure is needed.

### SUMMARY OF THE INVENTION

Embodiments hereof describe a radiopaque marker including a core wire having a proximal portion and a distal portion, and a coil wrapped around the distal portion of the core wire. The core wire is formed from a shape memory material and the coil is formed from a radiopaque material. The radiopaque marker includes a delivery configuration wherein the radiopaque marker is substantially elongated and a deployed configuration wherein the distal portion of the radiopaque marker forms a substantially helical tube.

In a method for taking an image of a vessel, a radiopaque marker in a delivery configuration is advanced endoluminally into the vessel, wherein the delivery configuration is a substantially elongate wire with a proximal portion and a distal portion. The distal portion of the marker is radiopaque. Upon reaching the target vessel, the distal portion of the marker is deployed such that the distal portion forms of helical, tubular shape conforming to the walls of the vessel. An image of the vessel, such as a fluorographic image, is taken while the radiopaque marker is deployed in the vessel.

In a method for creating an in situ fenestration in a stent graft, a radiopaque marker is advanced in a delivery configuration endoluminally into a branch vessel. The delivery configuration of the marker is a substantially elongate wire with a proximal portion and a distal portion, wherein the distal portion is radiopaque. The marker is deployed in the branch vessel such that the distal portion forms a helical tube abutting the walls of the branch vessel. A primary stent graft is advanced into a primary vessel from which the branch vessel branches and the primary stent graft is deployed in the primary vessel. A piercing device is advanced endoluminally through a lumen of the primary stent graft and adjacent the branch vessel. The puncturing device is advanced through the wall of the primary stent graft in a direction aligned with the orientation of the branch vessel.

### BRIEF DESCRIPTION OF THE DRAWINGS

Embodiments will be further explained with reference to the accompanying drawings, which are incorporated herein and form a part of the specification. The drawings further serve to explain the principles of the invention and to enable a person skilled in the pertinent art to make and use the invention. The drawings are not to scale.

FIG. 1 is a schematic illustration of an embodiment of a radiopaque, shape memory marker in an elongated configuration.

FIG. 2 is a cross-sectional view of the marker of FIG. 1 taken along line 2-2.

FIG. 3 is a schematic illustration of the marker of FIG. 1 in a deployed, helical configuration.

FIG. 4 is a schematic cross-sectional view of the marker of FIG. 1 disposed in a sheath.

FIG. 5 is a schematic cross-sectional view of the marker and sheath of FIG. 4 with the sheath partially retracted.

FIG. 6 is a schematic illustration of another embodiment of a radiopaque, shape memory marker in an elongated configuration.

FIG. 7 is a schematic illustration of the marker of FIG. 6 in a deployed, helical configuration.

FIGS. 8-17 are schematic illustrations of steps in a method of using the marker of FIG. 1 in an endoluminal stent graft delivery and deployment procedure.

FIGS. 18-20 are schematic illustrations of steps in a method of using the marker of FIG. 1 in an endoluminal stent graft delivery and deployment procedure.

FIG. 21 is a schematic illustration of another embodiment of a radiopaque, shape memory marker in an elongated configuration.

FIG. 22 is a schematic illustration of the marker of FIG. 21 in a deployed, helical configuration.

### DETAILED DESCRIPTION

With reference to the accompanying figures, wherein like components are labeled with like numerals throughout the figures, illustrative radiopaque, shaped memory, helical markers and methods for their use are disclosed.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician.

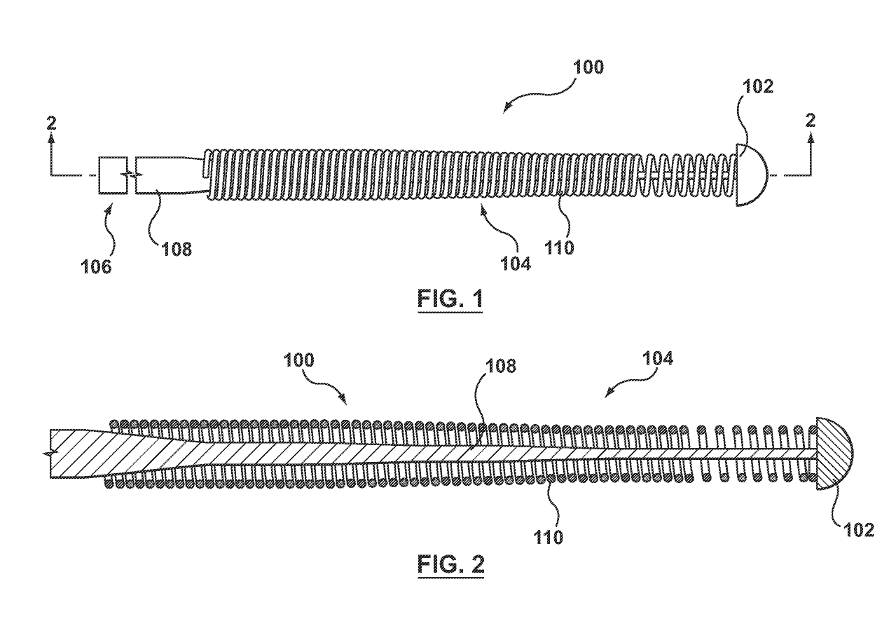

Referring now to the FIGS. 1-5, wherein components are labeled with like numerals throughout the several figures, an embodiment of a radiopaque marker 100 is shown. Marker 100 is shown in FIGS. 1-2 in an elongated, delivery, or non-deployed configuration. Marker 100 in this embodiment is similar in shape and construction to a guide wire, as known to those skilled in the art. In particular, marker 100 includes a distal tip or weld 102, a distal portion 104, and a proximal portion 106. Distal portion 104 includes a core wire 108 and a coil 110 wrapped around core wire 108. Proximal portion 106 includes core wire 108. As best seen in FIG. 2, core wire 108 tapers from a first diameter at the proximal portion 106 to a second, smaller diameter at the distal portion 104. Coil 110 may be approximately 0.002-0.004 inch in diameter and may be secured to core wire 108, such as by brazing, welding, or other means known to those skilled in the art, at a proximal connection. The distal end of the coil 108 may be secured to the distal end of core wire 108, such as by brazing, welding, or other means known to those skilled in the art. This commonly known construction for a guide wire provides pushability of the proximal portion 106 and flexibility of distal portion 104 for navigating tortuous vessels, as known to those skilled in the art. Configurations such as the one shown in FIGS. 1-2 are well known in the art for guide wires and are described in more detail, for example, in U.S. Pat. No. 4,545,390 to Leary and U.S. Pat. No. 4,922,924 to Gambale et al., both of which are incorporated by reference in their entireties herein. Many other configurations of the core wire and coil are known to those skilled in the art of guide wires, and can be utilized in the marker of the present invention. Marker 100 may be approximately 0.014 inch in diameter, a common size for guide wires. Other sizes for marker 100 may be utilized depending particular application, as known to those skilled in the art.

Core wire 108 is made of shape memory material. A shape memory material is capable of being deformed by an applied stress, and then recovering to its original unstressed shape. The shape memory material may exhibit thermoelastic behavior so that core wire 108 will transform to the original unstressed state upon the application of a stimulus, such as heat. The shape memory material may also exhibit stress-induced martensite, in which the martensite state is unstable and core wire 108 transforms back to the original state when a constraint has been moved, such as a sheath or guiding catheter described below. Suitable shape memory materials for core wire 108 include, but are not limited to, nickel-titanium alloys (i.e., Nitinol™), annealed platinum, annealed stainless steel, copper-zinc alloys, copper-aluminum alloys, copper-zinc-aluminum alloys, copper-aluminum-nickel alloys, and other alloys known to those skilled in the art. Non-metal shape memory materials, such as the polymer oligodial, may also be used, provided that the selected material is configured to have sufficient stiffness at proximal portion 106 for pushability through the vasculature or is attached to a stiffer material at proximal portion 106.

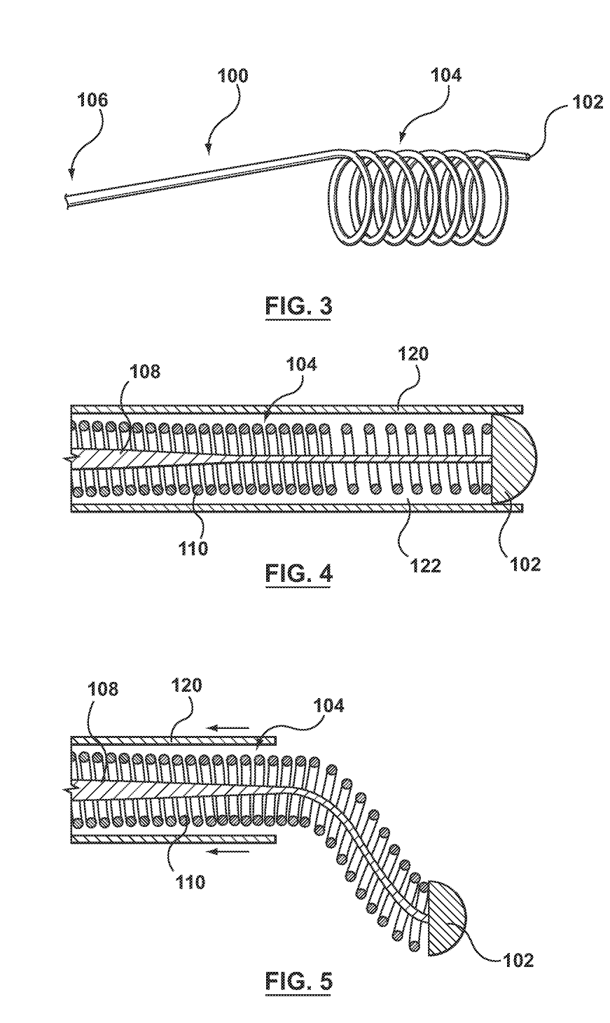

A substantially helical, tubular shape is placed on core wire 108 during manufacture, as shown in FIG. 3. Core wire 108 may be deformed into the delivery configuration of FIG. 1 by a restraining force, such as a sheath, or may be returned to the simple wire shape of FIG. 1 until activated by heat or electrical stimulus to assume the “remembered” substantially helical, tubular shape.

Commonly used shape memory materials, such as Nitinol™, may not be sufficiently radiopaque to be seen clearly under fluoroscopy. According, coil 110 is made from a radiopaque material. For example, and not by way of limitation, coil 110 may be made from platinum, gold, tungsten, iridium, tantalum, thallium or other materials known to those skilled in the art. Coil 110 may be made from a material that is more radiopaque than the material of core wire 108.

As noted briefly above, FIG. 3 shows distal portion 104 of marker 100 in a deployed configuration. The deployed configuration is a substantially helical tube. The diameter of the helical tube may be slightly larger than the diameter of the target vessel. For example, and not by way of limitation, a marker 100 used in a renal artery, as described in more detail below, may have an unconstrained diameter of approximately 9 mm in the deployed configuration (renal arties are generally 5-7 mm in diameter). As shown in FIG. 3, proximal portion 106 of marker 100 remains in a generally elongated configuration. This may result from proximal portion 106 remaining in a constraining sheath or tube, proximal portion 106 not being annealed into a helical tube shape, core wire 108 of proximal portion 106 being a different material from core wire 108 of distal portion 104, or other reasons known to those skilled in the art.

FIGS. 4-5 show distal portion 104 of marker 100 disposed in a sheath or guide catheter 120 having a lumen 122. Sheath 120 may be any suitable tube, preferably as flexible as possible while having sufficient rigidity to restrain marker 100 in the elongated, non-deployed configuration. Sheath 120 may be made of materials commonly used for guide catheters. For example, and not by way of limitation, polymeric materials such as polyethylene, polypropylene, polyurethane, and polyesters may be used. Other materials or combination of materials may be selected to provide the optimum balance of stiffness and flexibility. When sheath 120 is retracted, as beginning to show in FIG. 5, distal portion 104 of marker 100 returns to the helical, tubular shape set during manufacture.

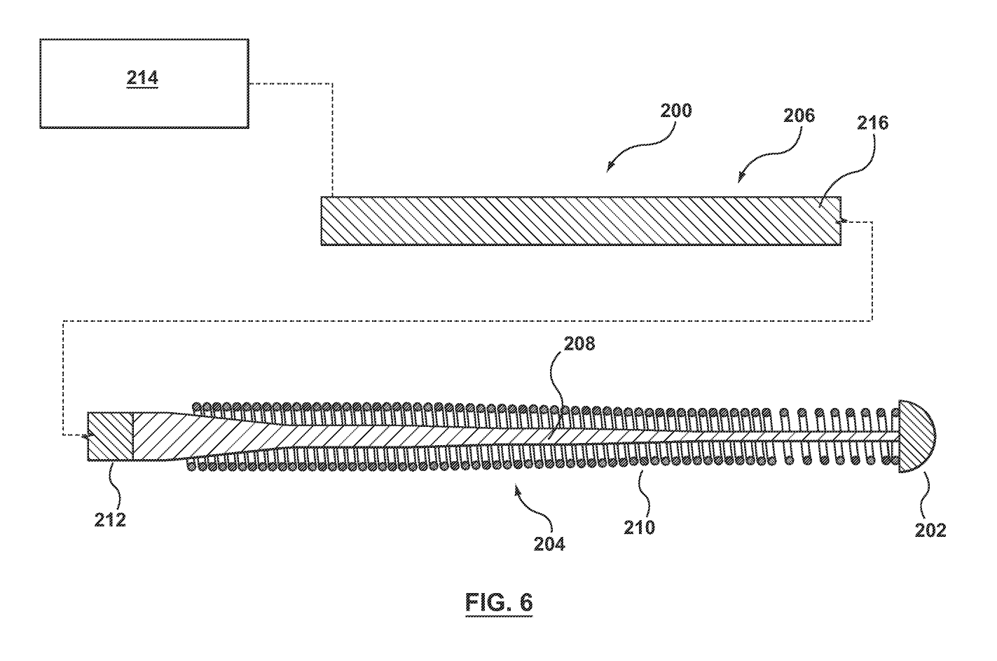

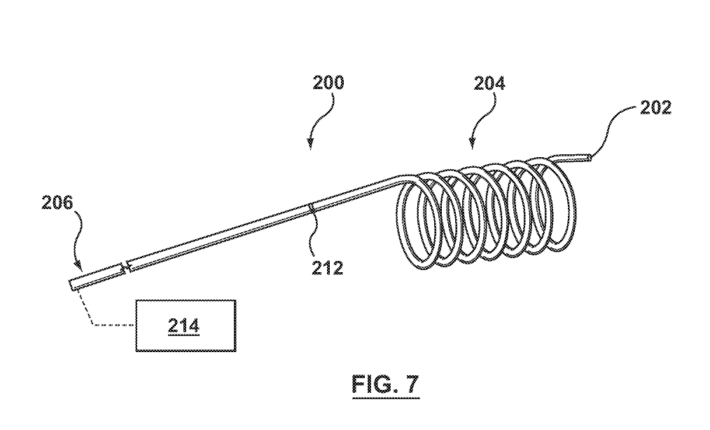

FIGS. 6-7 show another embodiment of a marker 200. Marker 200 is similar to marker 100 except that an outside stimulus 214, such as electricity or heat, is used to deploy marker 200. Marker 200 includes a distal tip or weld 202, a distal portion 204, and a proximal portion 206. Distal portion 204 includes a distal core wire 208 and a coil 210. Coil 210 is made from a radiopaque material, as described above with respect to marker 100. Distal core wire 208 is made from a shape memory material that returns to its preformed shape (helical tube) upon activation by stimulus 214. Proximal portion 206 is made of a proximal core wire 216 that is attached to core wire 208 at connection 212. Connection 212 may be a weld, adhesive, fusion, or other connection known to those skilled in the art. Proximal core wire 216 is a material that is not affected by outside stimulus 214. In another embodiment, proximal core wire 216 and distal core wire 208 may be a single, unitary body made of the same material, but only distal core wire 208 is pre-shaped to a substantially helical tube such that upon application of stimulus 214, only distal core wire 208 deploys to the substantially helical tubular shape shown in FIG. 7.

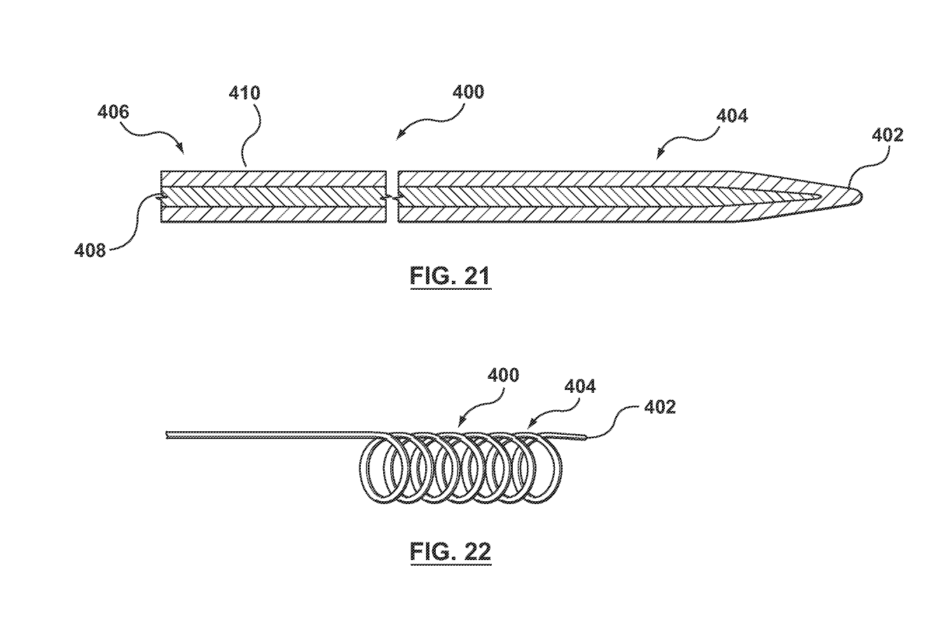

FIGS. 21-22 show another embodiment of a marker 400. Marker 400 is shown in FIG. 21 in an elongated, delivery, or non-deployed configuration. Marker 400 in this embodiment is similar in shape and construction to a guide wire such as the Glidewire® available from Terumo Interventional Systems. In particular, marker 400 includes a distal tip 402, a distal portion 404, and a proximal portion 406. Marker 400 includes a core wire 408 and a polyurethane jacket 410 surrounding core wire 408. Marker 400 may further include a hydrophilic coating (not shown). Core wire 408 tapers from a first diameter at the proximal portion 406 to a second, smaller diameter at the distal portion 404. As in the embodiments described above, core wire 408 is made of shape memory material, such as nickel-titanium alloys (i.e., Nitinol™), annealed platinum, annealed stainless steel, copper-zinc alloys, copper-aluminum alloys, copper-zinc-aluminum alloys, copper-aluminum-nickel alloys, and other alloys known to those skilled in the art. Non-metal shape memory materials, such as the polymer oligodial, may also be used, provided that the selected material is configured to have sufficient stiffness at proximal portion 406 for pushability through the vasculature or is attached to a stiffer material at proximal portion 406.

A substantially helical, tubular shape is placed on core wire 408 during manufacture, as shown in FIG. 22. Core wire 408 may be deformed into the delivery configuration of FIG. 21 by a restraining force, such as a sheath, or may be returned to the simple wire shape of FIG. 21 until activated by heat or electrical stimulus to assume the “remembered” substantially helical, tubular shape. Jacket 410 includes radiopaque material dispersed throughout, such that jacket 410 is sufficiently radiopaque to be seen clearly under fluoroscopy. For example, and not by way of limitation, jacket 410 may include platinum, gold, tungsten, iridium, tantalum, thallium or other radiopaque materials known to those skilled in the art dispersed therein to provide sufficient radiopacity to marker 400. FIG. 21 shows a portion of marker 400 in a deployed configuration. The deployed configuration is a substantially helical tube. The diameter of the helical tube may be slightly larger than the diameter of the target vessel, as described above.

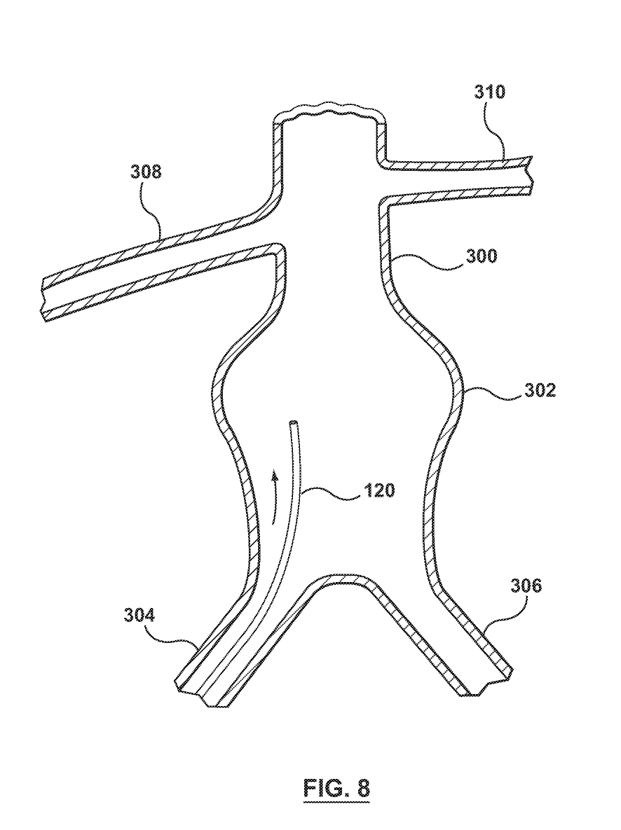

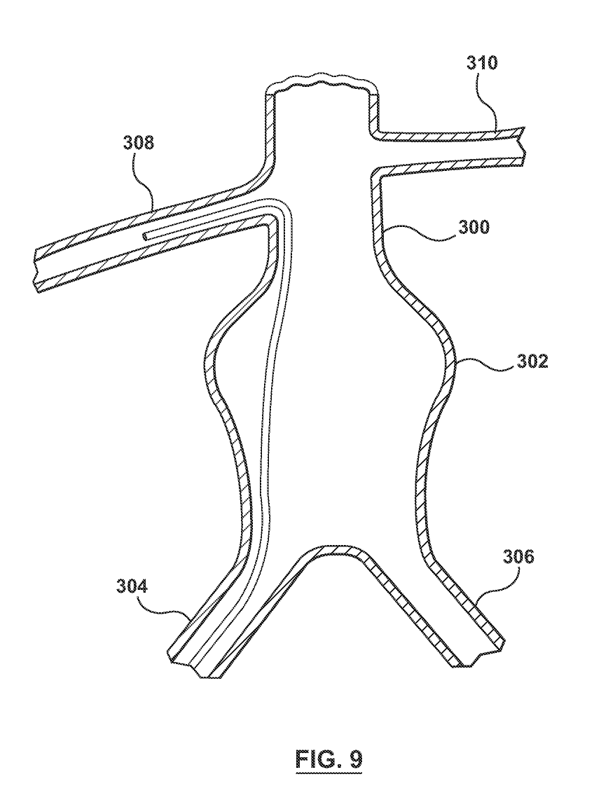

FIGS. 8-17 show some steps of an embodiment of a using marker 100 in an endoluminal stent graft placement procedure. In particular, the procedure shown and described is for placement of a primary stent graft in the abdominal aorta and a branch stent graft in a renal artery. However, the use of marker 100 is not limited to such a procedure, and can be used in any procedure wherein improved visualization of the position, orientation, and overall geometry of a vessel is desirable. Further, although the method is described with respect to marker 100, those skilled in the art would understand that marker 200, marker 400, or other variations of markers that form a helical tube in the vessel, may be used. FIGS. 8-17 show schematically an abdominal aorta 300 that includes an aneurysm 302. Also shown are the right common iliac artery 304, the left common iliac artery 306, the right renal artery 308, and left renal artery 310. The figures show views of the abdominal aorta from the front of the body, such that the right renal artery 308, for example, is shown on the left in the figures because the right renal artery is on the right of the body from the perspective of the person to whom the body belongs.

Referring to FIG. 8, sheath 120, with marker 100 disposed therein, is advanced from the femoral artery (not shown), through right iliac artery 304, and into abdominal aorta 300. As would be understood by those skilled in the art, sheath 120 and marker 100 may be advanced through other vessels, such as the left iliac artery 306. Sheath 120 and marker 100 are further advanced into the right renal artery 308. Distal tip 102 of marker 100 may shaped as known to those skilled in the art, and may be disposed distal of a distal end of sheath 120 to assist in guiding marker 100 and sheath 120 through the vasculature and into right renal artery 308. Those skilled in the art would understand that if marker 200 is utilized, sheath 120 may not be necessary.

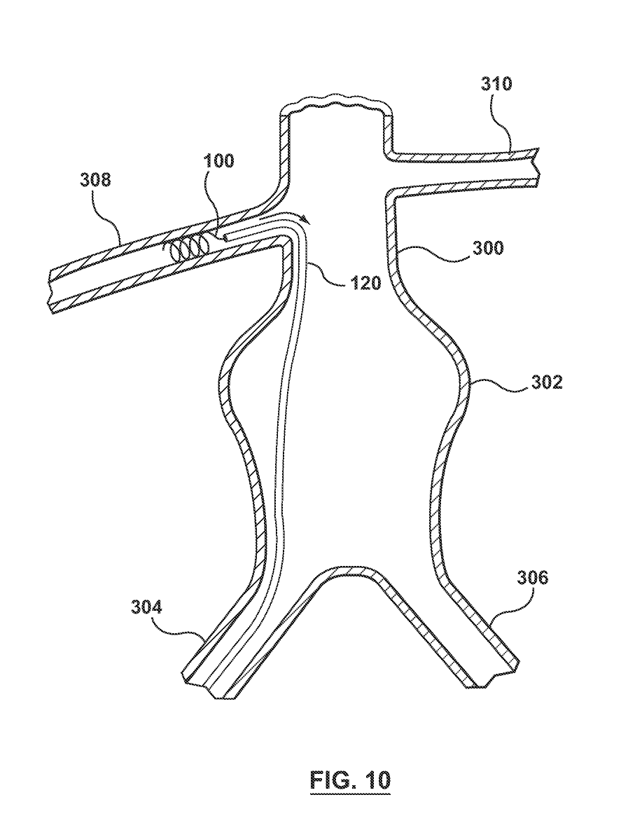

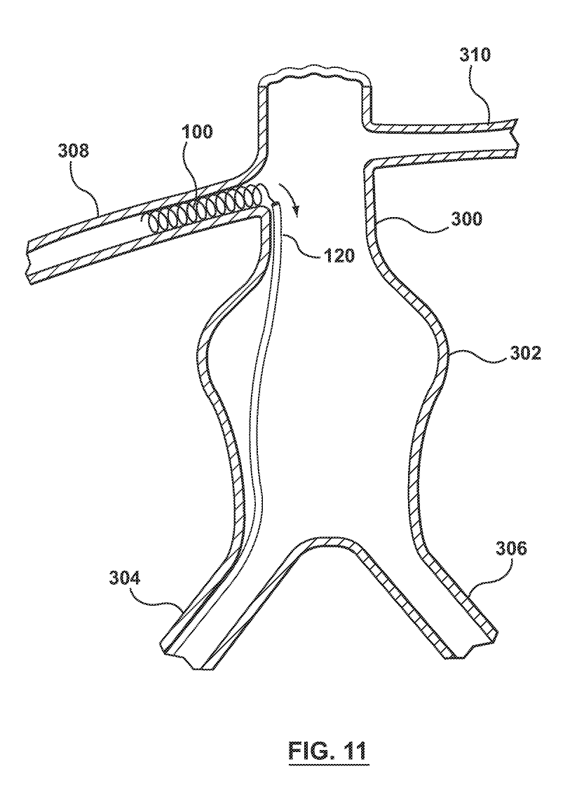

Sheath 120 is then retracted, as shown in FIGS. 10-11. As sheath 120 is retracted, marker 100 returns to the helical tubular shape set during manufacture. As described above, marker 100 is radiopaque and is now deployed against the walls of right renal artery 308. Accordingly, fluoroscopic images taken while marker 100 is deployed enable the clinician to visualize the position, orientation, and overall geometry of the right renal artery 308 in which marker 100 is deployed. Further, three-dimensional models of the right renal artery 308 may be produced in real-time or near real-time. For example, and not by way of limitation, Seimens Axiom Artis fluoroscopy systems are capable of rotary acquisition of multiple fluoroscopic images. The x-ray source and the flat panel detector on the c-arm rotate about the target. The software in the system then reconstructs a 3D virtual image of any radiopaque structures. The operator can “erase” interfering structures (like bone). A useful image can be created in less than a minute. Such improved visualization over conventional individual marker points enables the clinician to better place the primary stent graft, more accurately delivery the branch vessel guide wire, and more safely provide in situ fenestrations through the primary stent graft, as described in more detail below. Those skilled in the art would understand that the step of deploying marker 200 would be to apply the outside stimulus 214 to marker 200.

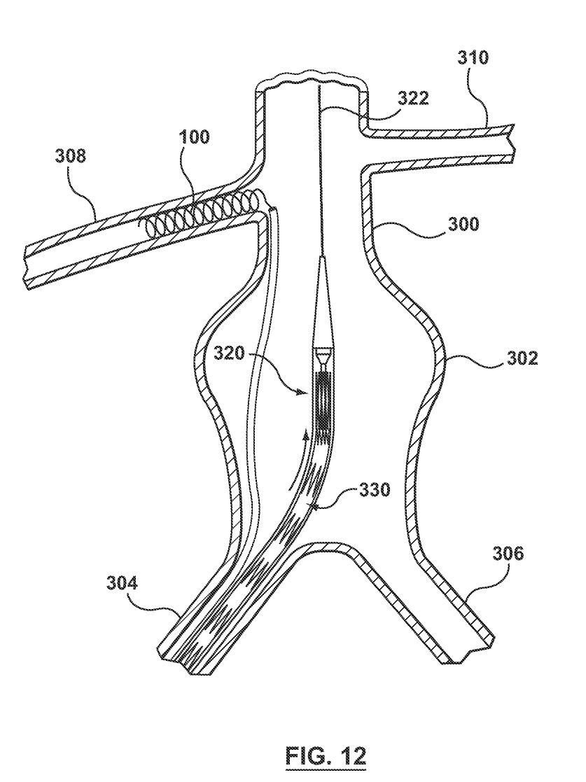

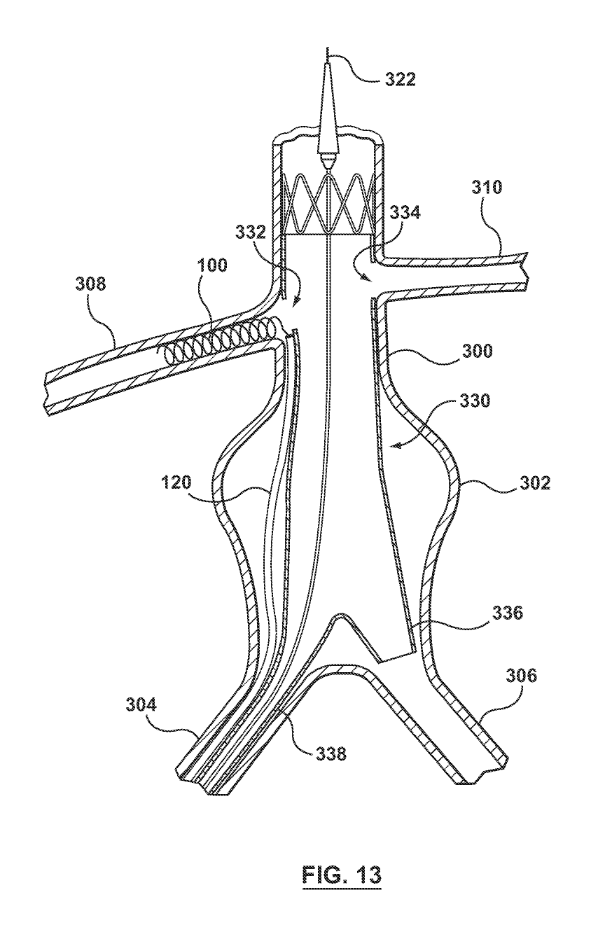

After marker 100 is deployed, a primary vessel guide wire 322 is advanced into the abdominal aorta 300, and a catheter delivery system 320 with a primary stent graft 330 is advanced over guide wire 322 into the abdominal aorta, as shown in FIG. 12. Delivery system 320 and primary stent graft 330 can be any delivery system and graft known to those skilled in the art. Marker 100 assists in proper placement of primary stent graft 330 relative to right renal artery 308 and alignment of fenestration 332 (shown in FIG. 13) with right renal artery 308. As shown in FIG. 13, when primary stent graft 330 is deployed, sheath 120 remains outside of primary stent graft 330. Primary stent graft 330 may also include a mobile external coupling configured to pop-out from primary stent graft 330 and extend into renal artery 308 for sealing with a branch vessel stent graft, as described, for example, in U.S. application Ser. No. 12/425,628 (filed Apr. 17, 2009 and published as U.S. 2010/0268319); Ser. No. 12/425,616 (filed Apr. 17, 2009 and published as U.S. 2010/0268327); Ser. No. 12/770,536 (filed Apr. 29, 2010); and Ser. No. 12/770,566 (filed Apr. 29, 2010). Deployed marker 100 assists in visualization of right renal artery 308 such that a mobile external coupling as described in the above applications may be properly aligned with and extended into renal artery 308.

Depending on the type of graft utilized, additional steps for deploying primary stent graft 330, such as deployment of an integral leg 338, and delivery and deployment of an extension leg 340 coupled to a short leg 336, are also contemplated, as known to those skilled in the art.

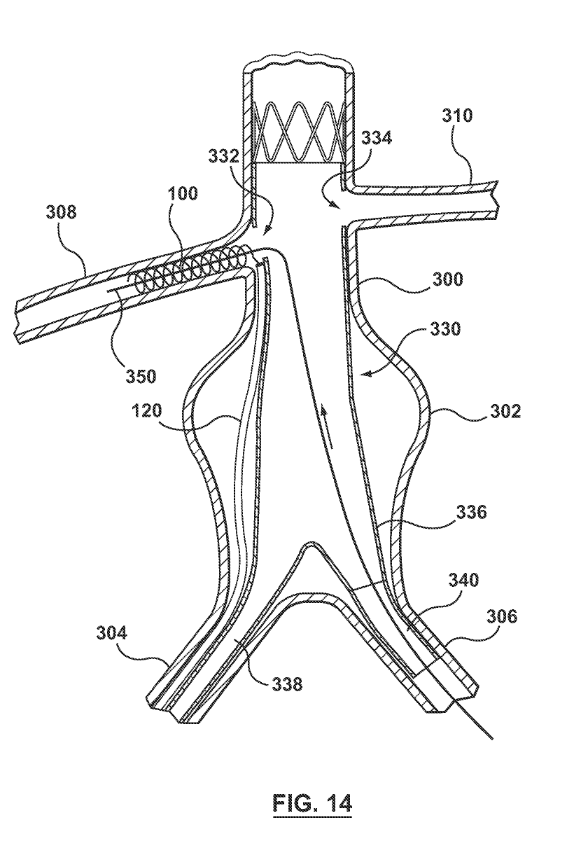

Delivery system 320 for primary stent graft 330 may be removed, and guide wire 350 for delivery of a branch vessel stent graft is advanced into right renal artery 308, as shown in FIG. 14. Marker 100 assists in visualization of right renal artery 308 for accurate delivery of guide wire 350. Although guide wire 350 is shown being delivered through the left iliac artery 306, those skilled in the art would recognize that guide wire 350 can be advanced along the same path as guide wire 322. Further, after delivery system 320 is removed, guide wire 322 could be partially retracted and then advanced into right renal artery 308 instead of utilizing guide wire 350.

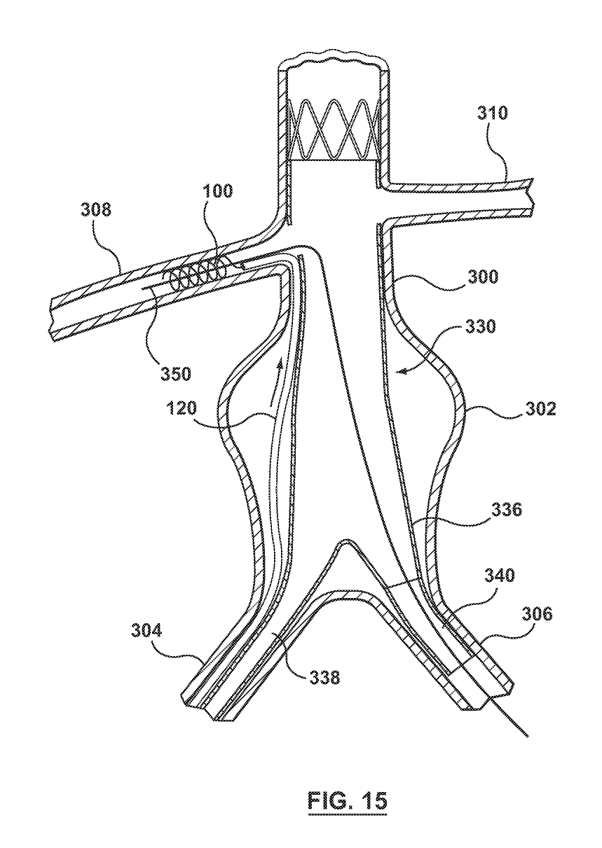

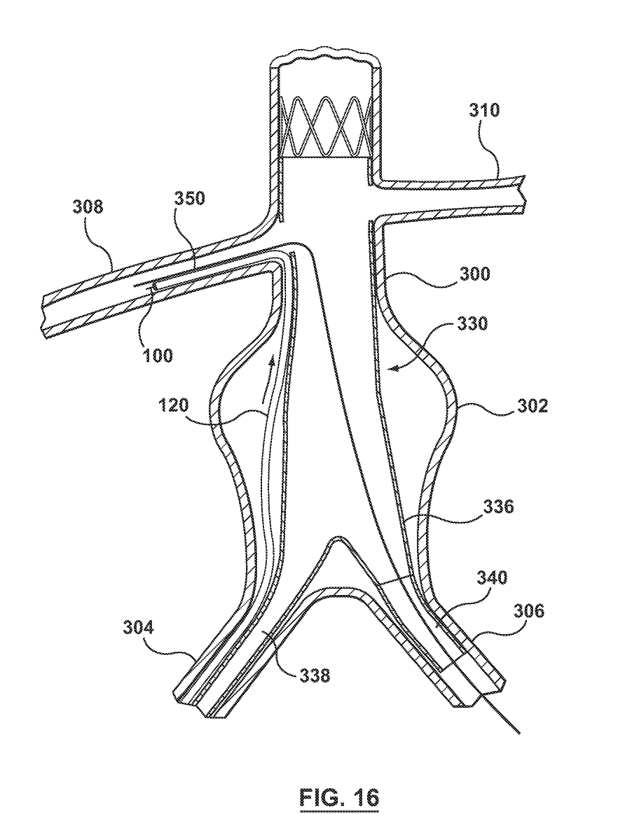

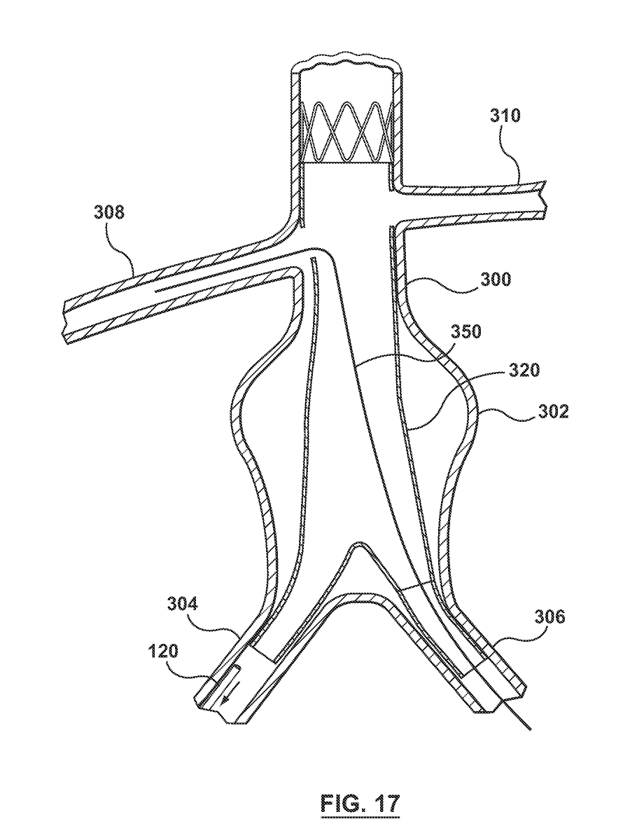

Sheath 120 is then advanced distally relative to marker 100, as shown in FIGS. 15-16, to recapture marker 100 within sheath 120. As sheath 120 is advanced, sheath 120 overcomes the pre-set helical tubular shape of distal portion 104 of marker 100 to capture marker 100 within sheath 120. Sheath 120 and marker 100 are then retracted proximally out of the body, as shown in FIG. 17. If marker 200 is utilized, removal of stimulus 214 returns marker 200 to its elongated configuration for removal from the vasculature. Further steps, such as delivery and deployment of a branch stent graft (not shown) into right renal artery 308, may then be performed.

The method described above was described using right renal artery 308. Those skilled in the art would understand that marker 100 can be used equally in left renal artery 310, and that markers can be used in both renal arteries simultaneously.

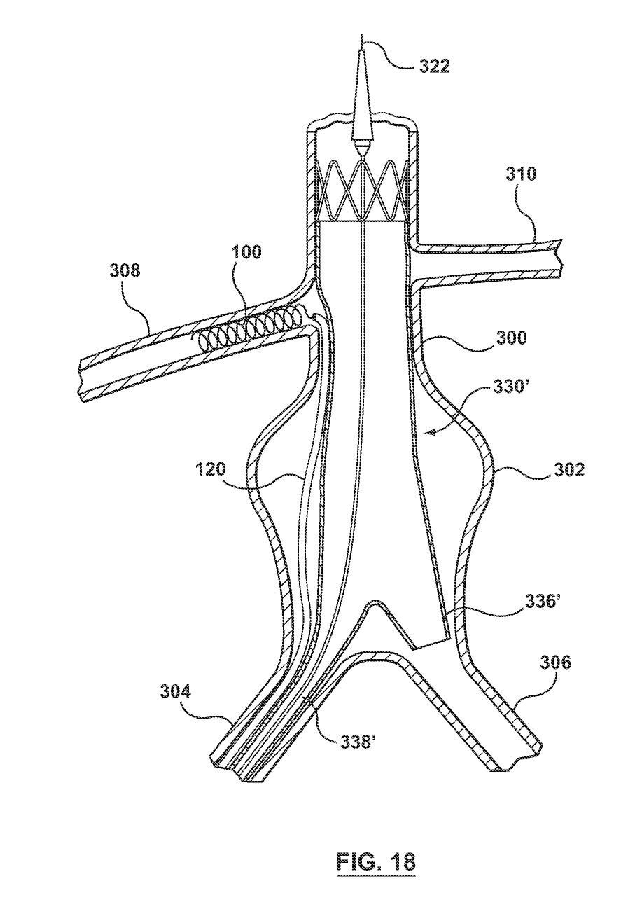

FIGS. 18-20 show some of the steps in an embodiment for using marker 100 to assist in forming in situ fenestrations in a primary stent graft. Marker 100 may be advanced and deployed within right renal artery 308 in the same manner described above with respect to FIGS. 8-11. A primary stent graft 330′ may then be advanced and deployed within abdominal aorta 300, as described above with respect to FIGS. 12-13, and shown in FIG. 18. However, primary stent graft 330′ does not include fenestrations for branch vessels, in particular, for renal arteries 308, 310. The use of non-fenestrated stent grafts is desirable due to variations in the axial location, axial angle, and radial angle of the branch vessels from patient to patient.

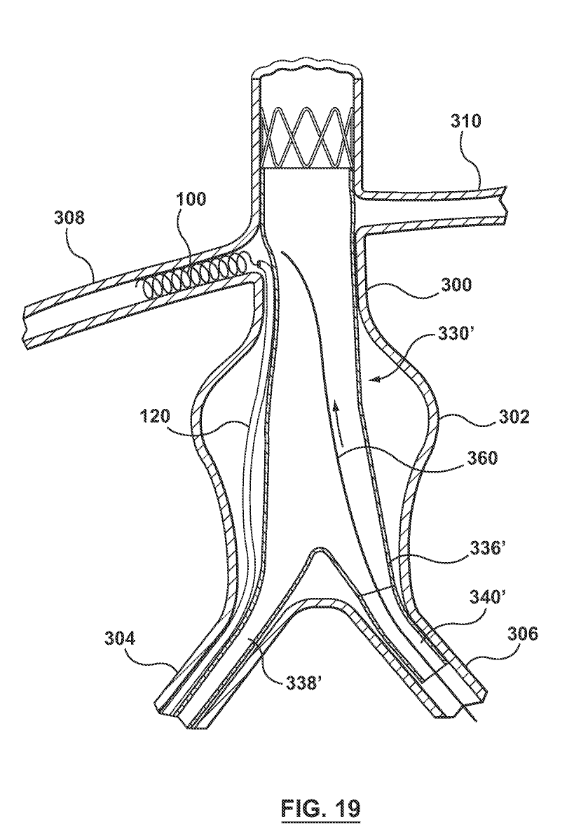

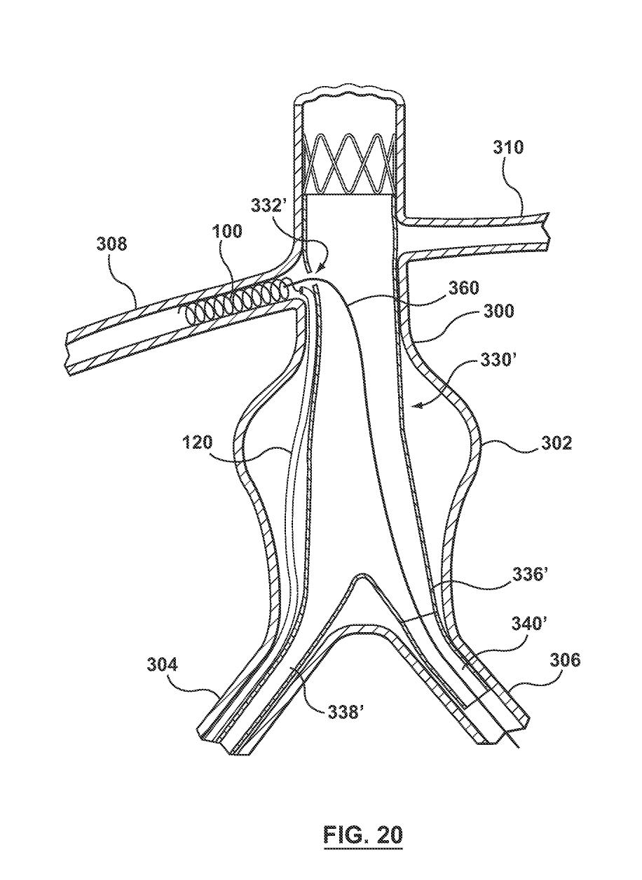

Accordingly, a puncturing device 360 for forming a fenestration through primary stent graft 330′ is advanced through primary stent graft to a location adjacent right renal artery 308, as shown in FIG. 19. Device 360 may be a needle, or may be similar to the devices described in U.S. Published Patent Application Publication No. 2008/0234717 to the same inventor hereof, or U.S. Published Patent Application Publication No. 2010/0106175 to McLachlan et al., or U.S. Published Patent Application Publication No. 2009/0125097 to Bruszewski et al., or may be an RF plasma catheter as described in U.S. Published Patent Application Publication Nos. 2009/0234348 to Bruszewski et al. and U.S. Published Patent Application Publication No. 2009/0264977 to Bruszewski et al., or an RF electrode as described in Published Patent Application Publication No. 2008/0108987 to Bruszewski et al., all of which are incorporated by reference herein in their entireties. Puncturing the graft material of primary stent graft 330′ may be dangerous if the device 360 is not properly aligned with right renal artery 308 in three dimensions (i.e., longitudinal and circumferential location, axial angle, and radial angle). For example, and not by way of limitation, a device 360 at the proper location for right renal artery 308 but advanced at the wrong angle may pierce a wall of branch vessel as it is advanced through the graft material of primary stent graft 330′. By utilizing marker 100, a real-time three dimensional image of the right renal artery 308 and device 360 enables the device 360 to be properly aligned with right renal artery 308 and then advanced through the graft material of primary stent graft 330′ to create a fenestration 332′ in situ, as shown in FIG. 20. Further steps for delivering and deploying a branch vessel stent graft in right renal artery 308 may then be performed, as known to those skilled in the art.

Although the description of FIGS. 18-20 was limited to the right renal artery 308, those skilled in the art would recognize that a separate marker could be advanced into left renal artery 310 such that both right and left renal arteries 308/310 may be visualized and in situ fenestrations may be provided through the primary stent graft and aligned with each. Further, although specifically described with respect to renal arteries, those skilled in the art would recognize that the markers described herein can be used to assist in providing in situ fenestrations through primary stent grafts deployed at other branch vessels.

The foregoing detailed description and examples have been given for clarity of understanding only. No unnecessary limitations are to be understood therefrom. It will be apparent to those skilled in the art that many changes can be made in the embodiments described.

What is claimed is:

1. A radiopaque marker comprising:
a core wire have a proximal portion and a distal portion, the core wire formed from a shape memory material; and
a coil wrapped around the distal portion of the core wire and coupled to the core wire,
wherein at least one of the core wire and the coil is formed from a radiopaque material,
wherein the core wire includes a delivery configuration wherein the core wire is substantially elongated and a deployed configuration wherein the distal portion of the core wire forms a substantially helical, tubular shape.

2. The radiopaque marker of claim 1, wherein the distal portion of the core wire is more flexible than the proximal portion of the core wire.

3. The radiopaque marker of claim 1, wherein the shape memory material is selected from the group consisting of nickel-titanium alloys, annealed platinum, annealed stainless steel, copper-zinc alloys, copper-aluminum alloys, copper-zinc-aluminum alloys, and copper-aluminum-nickel alloys.

4. The radiopaque marker of claim 1, wherein the radiopaque material is selected from the group consisting of platinum, gold, tungsten, and titanium.

5. The radiopaque marker of claim 1, wherein the coil is formed from the radiopaque material and is more radiopaque than the core wire.

6. A method for visualizing a vessel comprising the steps of:
advancing a radiopaque marker in a delivery configuration endoluminally into the vessel, wherein the delivery configuration is a substantially elongate wire with a proximal portion and a distal portion, wherein the distal portion is radiopaque;
deploying the radiopaque marker such that the distal portion forms a helical tube abutting walls of the vessel; and
taking an image of the vessel with the radiopaque marker deployed therein.

7. The method of claim 6, wherein the radiopaque marker comprises a core wire extending from the proximal portion to the distal portion, and a coil wrapped around the core wire at the distal portion.

8. The method of claim 7, wherein the core wire is formed from a shape memory material and the coil is formed from a radiopaque material.

9. The method of claim 7, wherein the shape memory material is selected from the group consisting of nickel-titanium alloys, annealed platinum, annealed stainless steel, copper-zinc alloys, copper-aluminum alloys, copper-zinc-aluminum alloys, and copper-aluminum-nickel alloys.

10. The method of claim 7, wherein the radiopaque material is selected from the group consisting of platinum, gold, tungsten, and titanium.

11. The method of claim 6, wherein the step of deploying the radiopaque marker comprises retracting a sheath surrounding the distal portion of the radiopaque marker.

12. The method of claim 6, wherein the step of deploying the radiopaque marker comprises applying a stimulus to the radiopaque marker.

13. The method of claim 12, wherein the stimulus is selected from the group consisting of electricity and temperature.

14. The method of claim 6, wherein the step of taking an image of the vessel comprises taking a fluorographic image.

15. The method of claim 6, further comprising the step of a making a three-dimensional model of the vessel based on the image.

16. A method for creating an in situ fenestration in a stent graft comprising the steps of:
advancing a radiopaque marker in a delivery configuration endoluminally into the branch vessel, wherein the delivery configuration is a substantially elongate wire with a proximal portion and a distal portion, wherein the distal portion is radiopaque;
deploying the radiopaque marker such that the distal portion forms a helical tube abutting walls of the branch vessel;
advancing endoluminally a stent graft into a primary vessel from which the branch vessel branches and deploying the stent graft in the primary vessel;
advancing a puncturing device endoluminally through a lumen of the primary stent graft and adjacent the branch vessel; and
advancing the puncturing device through a wall of the primary stent graft in a direction aligned with the orientation of the branch vessel.

17. The method of claim 16, further comprising the step of taking an image of the primary and branch vessels with the radiopaque marker deployed in the branch vessel and prior to the step of advancing the puncturing device through the wall of the primary stent graft.

18. The method of claim 17, wherein the step of taking an image comprises taking a fluorographic image.

19. The method of claim 17, further comprising the step of making a three-dimensional model of the branch vessel based on the image.
